(19) United States
(12) Patent Application Publication
(10) Pub. No.: US 2009/0131380 A1
(43) Pub. Date: May 21, 2009

(54) Novel pharmaceutical composition for topical treatment of skin psoriasis and the treatment method thereof

(76) Inventor: Pekka Heino, Helsinki (FI)

Correspondence Address: JACOBSON HOLMAN PLLC, 400 SEVENTH STREET N.W., SUITE 600, WASHINGTON, DC, 20004, US

(21) Appl. No.: 11/984,370

(22) Filed: Nov. 16, 2007

(51) Int. Cl. A61K 31/56; A61P 17/06

(52) U.S. Cl. 514/171

(57) ABSTRACT

The present invention provides the combination of topical 3,3,′5,5′-tetraiodothyroacetic acid and betamethasone for the treatment of skin psoriasis results in a better outcome than the combination of topical calcipotriol and betamethasone. The present invention also provide a method for treatment of skin psoriasis by using the composition of 3,3,′5,5′-tetraiodothyroacetic acid and betamethasone.

### FIELD OF THE INVENTION

The present invention relates to the scope of topical pharmaceutical treatments of the dermatological disease psoriasis.

### BACKGROUND OF THE INVENTION

Conventional topical treatments of skin psoriasis comprise uses of different hormonal compounds such as vitamin D derivatives (e.g. calcipotriol), retinoids (e.g. tazarotene) and glucocorticoids (e.g. betamethasone). None of these compounds are completely effective. They only reduce the size and thickness of the psoriatic skin lesions. Conventionally, the best pharmacological results have been achieved by the combination of calcipotriol and a glucocorticoid. The use of this combination is commonly recommended in current topical therapy of skin psoriasis. In fact, there is a commercial ointment containing both substances (calcipotriol and betamethasone) for the referred treatment (Daivobet®; LEO Pharma A/S, Ballerup, Denmark). However, the duration of treatment with the combination of these two antipsoriatic agents should not exceed four weeks due to probable side effects related to the concerned glucocorticoid. Even this combination does not result in the cure of psoriatic skin lesions, but usually significantly relieves the symptoms.

In current dermatological therapy, it is believed that thyroid hormones are not applicable for the purpose of treating psoriasis. However, several patents have been applied for the use of these compounds for the topical treatment of psoriasis. For instance, in international patent publication WO96/40048 (Inventors: T. Lavin and A. Vahquist), the entire exemplifying example on the topical effect of thyroid hormone compounds was based on a human experiment carried out with 3,3′5-triiodothyroacetic acid (also known by the names: “Triac, T3Ac, TA3 or T3A”), hereinafter “Triac”. The conclusion was that an antipsoriatic effect had emerged in 60% of the experiment persons treated with topical Triac (Page 30, lines 15-25 in WO96/40048, page 18, lines 31-40 in the corresponding European patent EP 0 831 769 B1 and column 15 in the corresponding U.S. Pat. No. 6,221,911 B1). However, in a more accurate clinical study conducted later by the inventor, A. Vahlquist himself, it turned out that this result did not hold: In this placebo-controlled double-blind study, which was carried out in 1997, Triac was found to be ineffective in the topical treatment of plaque psoriasis. The result was published by Vahlquist et al. in 2004 in Volume 151 (pages 489-491) of British Journal of Dermatology as a concise communication with the title: “Inefficacy of topical thyroid hormone analogue TriAc in plaque psoriasis: results of a double-blind placebo-controlled trial”. In another international patent publication WO011765589, and later in related U.S. continuation-in-part-application Ser. No. 11/198,133 (Inventor: P. Heino) the therapeutic use of two thyroid hormone compounds, 3,3′,5,5′-tetraiodothyroacetic acid (also known by the names Tetrac, T4Ac, TA4 or T4A), hereinafter “Tetrac”, and 3,3′,5,5′-tetraiodothyropropionic acid (also known by the names Tetraprop, TP4 or T4P), hereinafter “Tetraprop” in skin psoriasis is described. Differing strikingly from the ineffectiveness of Triac, Tetrac and Tetraprop was perceived to possess a strong clinical antipsoriatic efficacy, when administered topically in an amount of 50 μg/cm²/24 h. to the diseased skin in a vehicle (Locobase® unctuous creme, manufactured by Astellas Pharma A/S, Glostrup, Denmark) containing 0.05% (w/w) either Terac or Tetraprop, for 35 days. In U.S. Ser. No. 11/198,133, its was also shown again that corresponding topical 0.05% Triac (50 μg/g/cm²/24 h.) administered for an equal time to the diseased skin, was indeed ineffective in skin psoriasis. Another ineffective thyroid hormone compound in the topical treatment of skin psoriasis is introduced in the same document, too, namely D-thyroxine (page 10, line 30 to page 12, line 29). Despite their ineffectiveness in the topical treatment of skin psoriasis the mentioned compounds, Triac and D-thyroxine do not irritate or worsen psoriatic rash. However, topically administered L-thyroxine and L-triiodothyronine greatly worsen psoriatic rash, which phenomenon was shown earlier by practical clinical examples in WO1765589 and emphasized again in U.S. Ser. No. 11/198,133 (page 10, lines 8-11). WO96/40048 totally neglects this deleterious fact and teaches without reservation that besides Triac, D-thyroxine, L-thyroxine and L-triiodothyronine, a couple of hundreds naturally occurring and synthetic thyroid hormones and their derivatives are all suitable for the topical treatment of psoriasis and about twenty other skin diseases. In reality, this argument must be based merely on the inventors' arbitrary guesses.

Thus, is has been shown that the relevant exemplifying example 7 in WO/96/400048 is not enabled. It is also true that the example was wrong already at the moment of filing of the priority patent application of this invention. Hence, a clinical antipsoriatic effect of any thyroid hormone compound cannot be anticipated or predicted on the basis of WO/96/400048.

### SUMMARY OF THE INVENTION

An object of the present invention is to provide a method for treating skin psoriasis, which comprises administering a therapeutically effective topical amount of 3,3′5,5′-tetraiodothyroacetic acid and a glucocorticoid in combination with each other to the skin of a patient in need thereof.

Another object of the present invention is to provide a topical pharmaceutical composition for treating skin psoriasis, which contains 3,′3′,5,5′-tetraiodothyroacetic acid and a glucocorticoid, preferably betamethasone or its ester.

According to the present invention, besides the clinical antipsoriatic effect of an ointment containing Tetrac (hereinafter “the Tetrac-ointment”), a surprisingly more potent clinical antipsoriatic effect emerged when the Tetrac-ointment and an ointment containing a glucocorticoid, in this case betamethasone, was concurrently applied with the Tetrac-ointment to a psoriatic plaque. The therapeutic changes were verified by a series of high-resolution photographs and are presented below in the section of brief description of drawings. These results are clearly novel and surprising at least because the ultimate therapeutic effect was even better than that of a commercial ointment containing calcipotriol, applied alone or combined with the glucocorticoid, betamethasone.

### BRIEF DESCRIPTION OF DRAWINGS

FIG. 1 shows side by side photographs from top to bottom taken concurrently first about the untreated psoriatic elbow lesions (the first photograph pair), then taken in the same way after the treatment of two weeks (the second photograph pair), five weeks (the third photograph pair) and six weeks (the fourth photograph pair) comparing the results of the Tetrac regimen (left column) and the control regimen (right column).

FIG. 2 shows successive photographs taken about the lesion of the calf. From left to right on the top (first) row, the first photograph has been taken about the untreated position, the second one after the therapy of two weeks, the third one after the therapy of three weeks. Then, from right to left on the bottom (second) row, the first photograph has been taken after the therapy of four weeks, the second one after the therapy of five weeks and the third one after the therapy of six weeks.

### DESCRIPTION OF THE INVENTION

### Example

Patient

A psoriasis patient having almost symmetrical psoriatic plaques on the lateral sides of both elbows (l.dx: 41 cm² and l.sin: 44 cm²) and a psoriatic plaque (53 cm²) on his right calf was chosen for the trial.

Medicine

a) An ointment (Locobase® unctuous creme, Astellas Pharma A/S Glostrup, Denmark) in which 500 μg/g 3,3′,5,5′-tetraiodothyroacetic acid (0.05%) of GMP-quality (Pharmatory Ltd., Oulu, Finland) was mixed was used as an ex tempore preparation for the concerned treatment purpose (hereinafter referred to as “Tetrac”).

b) A 50 μg/g calcipotriol (0.005%) containing creme (Daivonex®, LEO Pharma A/S, Ballerup, Denmark) was available as commercial pharmaceutical product (hereinafter referred to as “Daivonex®”).

c) A betamethasone 17-valerate-containing creme (Bemetson®, Orion oyj, Espoo, Finland) responding betamethasone 1 mg/g (0.1%) was available as a commercial product (hereinafter referred to as “Bemetson®”).

Methodology

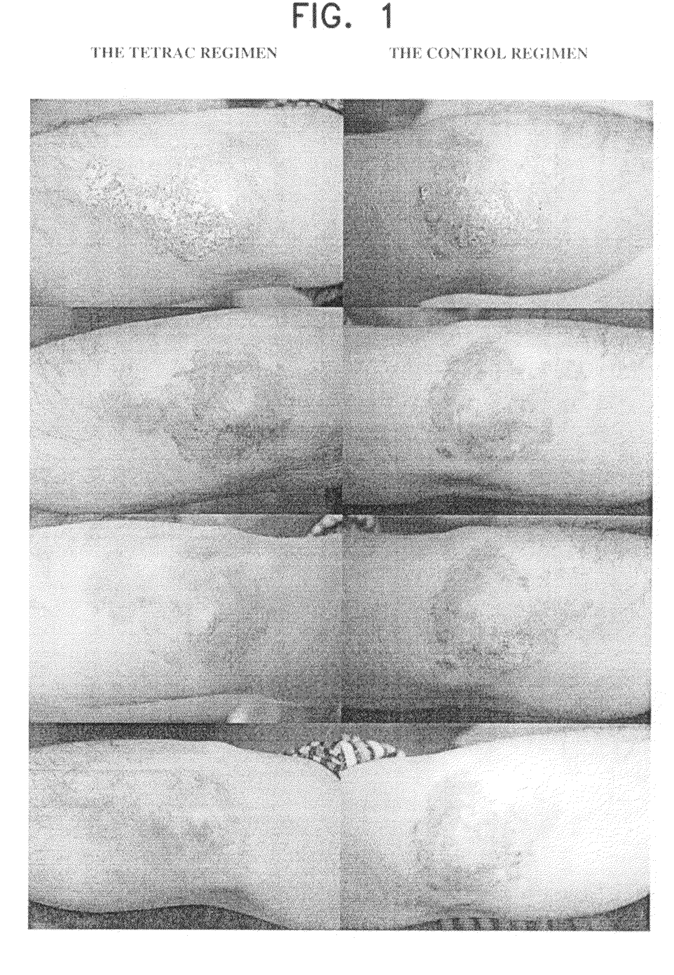

a) The psoriatic lesion of the left elbow of the patient was selected as the target of the topical treatment with Tetrac, the result of which is shown in left column of FIG. 1—“The Tetrac Regimen”.

b) The psoriatic lesion of the right elbow served as control, getting an equal amount of the unctuous creme Locobase® as the left elbow, but without 3,3′5,5′-tetraiodothyroacetic acid (100 mg of the mere unctuous creme twice a day, the result of which is shown in the right column of FIG. 1—“The Control Regimen”.

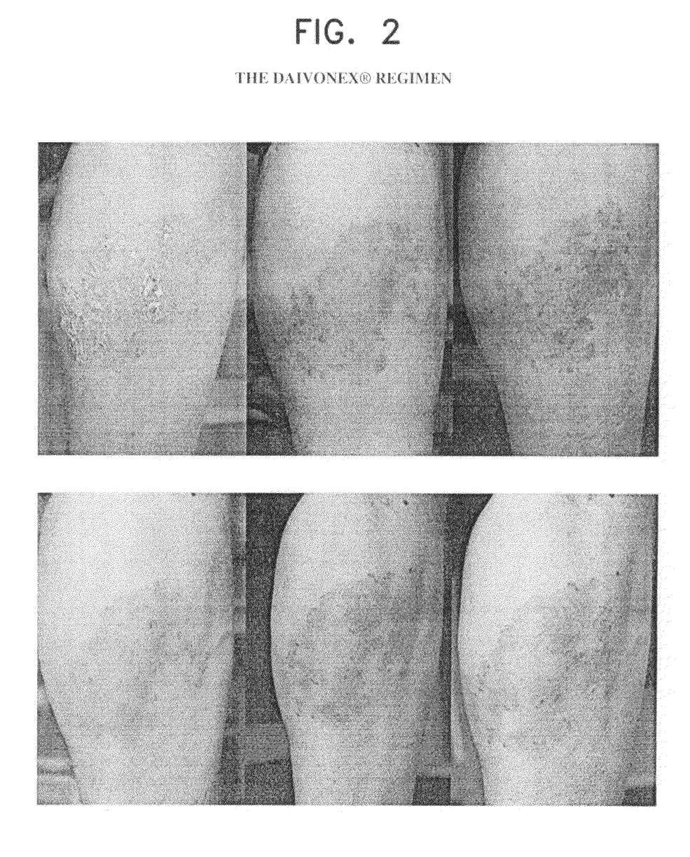

c) The psoriatic lesion of the right calf was selected as the target of the topical treatment with Daivonex®, the result of which is shown in FIG. 2—“The Daivonex® Regimen”.

100 mg of Tetrac (containing 50 μg 3,3′,5,5′-tetraiodothyroacetic acid (ca. 1.0 μg/cm²) was applied twice a day to the lesion of the left elbow and 150 mg of Daivonex® (containing 7.5 μg calcipotriol (ca. 0.15 μg/cm²) was applied twice a day to the lesion of the right calf for six weeks. For the last week of the six week experiment period, a concurrently applied dose of 100 mg of Bemetson® (containing 100 μg of betamethasone (ca. 2.0 μg/cm²) was added to the regimens, including the Control Regimen, twice a day. This can also be done by a single topical reparation composed of 3,3,′5,5′-tetraiodothyroacetic acid and the glucocorticoid.

Serum free thyroxine (S-T4f) and the pituitary thyroid stimulating hormone (S-TSH)-levels of the patient were monitored once a week and were stated to be within normal reference limits during the whole trial.

Results

As shown in FIG. 1, a clear clinical antipsoriatic effect of the topically applied Tetrac is seen after five weeks, and almost a complete healing of the psoriatic plaque treated for the final sixth week with Tetrac plus Bemetson® is seen at the end of the trial.

As shown in FIG. 2, none, or only a weak antipsoriatic effect is seen on the psoriatic lesion after the treatment of five weeks with Daivonex® and finally for the sixth week with Daivonex® plus Bemetson®.

It has to be pointed out that the “antipsoriatic effect” seen in the second photographs from top to bottom illustrating the Tetrac Regimen, The Control Regimen and the second photograph from left on the top line of the Daivonex® Regimen is mostly due to the mere disappearance of the scaling of the plaques, which has been taken place by the mechanical influence of washing the lesions with soap water and by the moisturizing influence of the vehicle.

The foregoing is considered as illustrative only of the principles of the invention. Further, since numerous modifications and changes will readily occur to those skilled in the art, it is not desired to limit the invention to the exact construction and operation shown and described, and, accordingly, all suitable modifications and equivalents may be resorted to, falling within the scope of the invention.

1. A method for treating skin psoriasis comprising administering a therapeutically effective topical amount of 3,3′5,5′-tetraiodothyroacetic acid and a glucocorticoid in combination with each other to the skin of a patient in need thereof.

2. The method according to claim 1, wherein the glucocorticoid is betamethasone or its ester.

3. The method according to claim 1, wherein the amount of 3,3′,5,5′-tetraiodothyroacetic acid is equal to or between 0.2 μg and 200 μg as applied to 1 cm² of psoriatic skin per 24 hours, and the amount of betamethasone or its ester applied to the same area of psoriatic skin responds an amount equal to or between 0.5 μg and 20 μg of betamethasone per 24 hours.

4. A topical pharmaceutical composition for treating skin psoriasis comprising 3,′3′,5,5′-tetraiodothyroacetic acid and a glucocorticoid, preferably betamethasone or its ester.
